(19) United States
(12) Patent Application Publication
(10) Pub. No.: US 2025/0231194 A1
(43) Pub. Date: Jul. 17, 2025

(54) METHOD FOR TRANSPLANT ORGAN HEALTH ASSESSMENT

(71) Applicant: BELGIAN VOLITION SRL, Isnes (BE)

(72) Inventor: Jacob Vincent MICALLEF, Isnes (BE)

(21) Appl. No.: 18/853,879

(22) PCT Filed: Apr. 6, 2023
(86) PCT No.: PCT/EP2023/059138

§ 371 Date: Oct. 3, 2024

(30) Foreign Application Priority Data
Apr. 8, 2022 (GB) 2205212.0

(51) Int. Cl. G01N 33/68; G01N 33/543

(52) U.S. Cl. CPC G01N 33/6875; G01N 33/543; G01N 2800/245; G01N 2800/50

(57) ABSTRACT

The invention relates to methods for assessing the health or suitability for transplant of a donor organ or tissue by measurement of extracellular traps (ETs).

### FIELD OF THE INVENTION

The present invention relates to cell free nucleosomes and extracellular traps as biomarkers for the assessment of extracorporeal organ degeneration, suitability of donor organs for transplantation and post-transplantation donor organ health.

### BACKGROUND OF THE INVENTION

Some 154,000 solid organ transplants are performed annually worldwide including 100,000 kidney, 36,000 liver, 9,000 heart and 7,000 lung transplants. In addition more than 85 million units of blood are transfused worldwide annually (Lotterman and Sharma. Blood Transfusion. In: StatPearls [Internet]. Treasure Island (FL): StatPearls Publishing; 2022 January).

The demand for organ transplant is greater than the supply of donated organs. In the USA the number of people on the waiting list for a kidney transplant was more than 90,000 persons in 2021, whilst approximately 25,000 kidney transplants were performed. In the meantime some 17 people on the waiting list died daily in 2021. In addition not all donated organs can be transplanted because many are found to be non-viable due to damage incurred extracorporeally. About 83% of donor kidneys are transplanted (Mohan et al. Kidney Int. 2018; 94 (1): 187-98) and only 40% of donated lungs and livers are transplanted (Snell et al. J. Heart Lung Transplant. 2008; 27 (6): 662-7; Orens et al. J. Heart Lung Transplant. 2003; 22 (11): 1183-200 and Zhai et al. Nat. Rev. Gastroenterol. Hepatol. 2013; 10 (2): 79-89). There is a need for better methods for the assessment of extracorporeal organ degeneration to identify organs which require treatment to improve or sustain their viability for transplant.

The mechanisms of chronic rejection are not fully understood, but it are thought to be related to vascular damage and tissue fibrosis. Due to the lack of mechanistic understanding the is a lack of biomarkers to predict rejection. The main predictors for the development of chronic rejection are therefore on clinical grounds and through tissue biopsy (Verhoeven et al. Clin. Epigenetics 2021; 13 (1): 32 and Krenzien et al. Front. Immunol. 2019; 10:758). Blood transfusion treatments may similarly suffer transfusion-related acute lung injury (TRALI).

To reduce or slow rejection, organ graft recipients receive immunosuppressive drug treatment for the whole lifetime of the transplanted organ. These treatments are associated with increased risk of infection and malignancy.

There is a need for informative biomarkers for immune responses in organ transplantation to identify rejection without invasive procedures.

### SUMMARY OF THE INVENTION

According to a first aspect of the invention, there is provided a method for the assessment of the health or suitability for transplant of a donor organ or tissue, wherein said method comprises the steps of:
(i) obtaining a fluid sample from the donor organ or tissue;
(ii) measuring the level of ETs or NETs in the sample; and
(iii) using the level of ETs or NETs measured as an indicator of the health of the donor organ or tissue or the suitability for transplant of the donor organ or tissue.

According to a further aspect of the invention, there is provided a method for the assessment of the health of a donor organ or tissue that has been transplanted in a subject wherein said method comprises the steps of:
(i) obtaining a plasma sample from the subject;
(ii) measuring the level of ETs or NETs in the plasma sample; and
(iii) using the level of ETs or NETs measured as an indicator of the health of a donor organ or tissue in the subject.

According to a further aspect of the invention, there is provided a method for the prediction or assessment of the risk of rejection of a donor organ or tissue that has been transplanted in a subject wherein said method comprises the steps of:
(i) obtaining a plasma sample from the subject;
(ii) measuring the level of ETs or NETs in the plasma sample; and
(iii) using the level of ETs or NETs measured as an indicator of the risk of rejection of the donor organ or tissue in the subject.

### BRIEF DESCRIPTION OF THE FIGURES

FIG. 1. The time course of NETosis in whole blood samples collected in serum tubes. Whole blood collected from 3 healthy volunteers in serum blood collection tubes was centrifuged at 30 minutes, 1 hour, 2 hours and 6 hours following venepuncture. The level of NETs produced was measured as the serum level of H3.1-nucleosomes. The assay response (Optical Density), corresponding to the serum H3.1-nucleosome level, rose with sample processing delay time indicating that any delay in sample processing will lead to false positive results for NETs and nucleosome measurements in serum.

FIG. 2. An investigation into NETosis in whole blood samples collected in EDTA plasma tubes. Whole blood collected from 3 healthy volunteers in EDTA blood collection tubes was centrifuged at 30 minutes, 1 hour, 2 hours, 4 hours, 6 hours and 24 hours following venepuncture. The level of NETs produced was measured as the plasma level of H3.1-nucleosomes. The assay response (Optical Density), corresponding to the plasma H3.1-nucleosome level, did not increase over 24 hours indicating that no NETosis occurred ex vivo in EDTA plasma samples and that EDTA plasma nucleosome measurements are a valid measure of circulating NETs and nucleosomes.

### DETAILED DESCRIPTION OF THE INVENTION

The present invention is related to the measurement of biomarkers for the assessment of the health of an organ for transplantation in the extracorporeal perfusate or an extracorporeal wash applied to the organ or other fluid derived of or from the organ.

When donor organs have been transplanted (or grafted) into a recipient host, there is a continued risk of organ degeneration or rejection within or the host body caused mainly by immune response from the host. Acute rejection occurring weeks or months following transplantation is thought to arise from recognition of major histocompatibility complex (MHC) proteins on the cell surfaces of the transplanted tissue by host lymphocytes leading to an immune response targeting the transplanted tissue. The MHC system (known as the human leukocyte antigen (HLA) system in humans) facilitates the immune recognition of self and it is extremely uncommon for any two subjects to have identical MHC alleles. Therefore MHC (or HLA) matching (in addition to matching blood groups) is critical in preventing acute rejection. Chronic rejection leads to organ (graft) degeneration over months or years following transplantation. Approximately 80% of lung transplants, 60% of heart transplants, 50% of kidney transplants and 10% of liver transplants are subject to chronic rejection 5 years post-transplantation, typically leading to organ failure around 10 years post-transplantation, or around 5 years for transplanted lungs.

There is currently a severe lack of biomarkers to assess the health of extracorporeal organs for transplant (Zhai et al. (2013) supra). This is largely due to the fact that there is a dearth of understanding of the nature of the injury present. The authors have deduced that a major cause of injury in isolated organs outside of the body is excessive production of extracellular traps (ETs), particularly neutrophil extracellular traps (NETs). Neutrophils may infiltrate the organs or tissues and remain present in the organ after removal from the body where they may produce excessive NETs within the organ or tissue. These high levels of NETs are toxic leading to cell death and loss of organ or tissue function. Severe loss of organ function leads to the organ being discarded for transplantation.

Therefore, according to a first aspect of the invention, there is provided a method for the assessment of the health or suitability for transplant of a donor organ or tissue, wherein said method comprises the steps of:
(i) obtaining a fluid sample from the donor organ or tissue;
(ii) measuring the level of ETs or NETs in the sample; and
(iii) using the level of ETs or NETs measured as an indicator of the health of the donor organ or tissue or the suitability for transplant of the donor organ or tissue.

References herein to “donor organ” or “donor tissue” refer to the extracorporeal organ or tissue that is intended for transplant into a recipient subject. Once the donor organ or tissue has been transplanted it may also be referred to as the “grafted organ” or “grafted tissue”.

Donor organs are typically sustained during storage prior to transplantation using extracorporeal perfusion with plasma from the donor or a perfusate comprised of a mixture of buffer and donor plasma. Following this, the organ may be perfused with whole blood obtained from the transplant recipient.

Therefore, in one embodiment the fluid sample from the donor organ or tissue to be tested for ETs is a plasma sample or other fluid sample obtained from the perfusate used to perfuse the donor organ. In other embodiments, the method additionally comprises applying a wash to the donor organ or tissue, and the fluid sample may be obtained from the wash applied or brought into contact with the organ. Therefore, in a further embodiment the fluid sample may be a fluid medium (e.g. wash) used to bathe the donor organ or tissue.

Transfusion related acute lung injury (TRALI) is a complication of the transfusion of donor whole blood, plasma or platelets typically leading to hypoxia and bilateral pulmonary edema that occurs in approximately 1 in 5000 units of transfused blood and has a 5% mortality rate. Therefore in one embodiment the extracorporeal tissue is whole blood or a blood product. The method may be used to ensure the (donor) blood does not contain excessive NETs levels in order to prevent TRALI.

It will be clear to those skilled in the art that assessment of the health of a donated organ or other tissue may be continued post transplantation in the recipient subject as an indicator of graft rejection or risk of graft rejection. Therefore, according to a further aspect of the invention, there is provided a method for the assessment of the health of a donor organ or other tissue that has been transplanted in a subject wherein said method comprises the steps of:
(i) obtaining a plasma sample from the subject;
(ii) measuring the level of ETs or NETs in the plasma sample; and
(iii) using the level of ETs or NETs measured as an indicator of the health of the donor organ or tissue in the subject.

References herein to “graft rejection” (which may also be referred to as “transplant rejection”) encompasses both acute and chronic transplant rejection.

In another aspect, there is provided a method for the prediction or assessment of the risk of rejection of a donor organ or tissue that has been transplanted in a subject, wherein said method comprises the steps of:
(i) obtaining a plasma sample from the subject;
(ii) measuring the level of ETs or NETs in the plasma sample; and
(iii) using the level of ETs or NETs measured as an indicator of the risk of rejection of the donor organ or tissue in the subject.

The donor organ (i.e. the organ to be, or that has been, transplanted) may be any organ including, without limitation, a kidney, liver, heart, lung, pancreas, stomach and intestine. Other donor organs and tissues include blood, plasma, platelets, cornea, bone, tendon, skin, pancreas islets, heart valves, nerves, veins, bone marrow, stem cells as well as limbs including hands, arms and feet.

Levels of cell-free nucleosomes have been investigated in serum samples obtained from kidney transplant recipients as biomarkers for kidney transplant rejection (Verhoeven et al. (2021) supra) and acute rejection was associated with elevated serum nucleosome levels and an area under the curve (AUC) of 0.73 in a receiver operating characteristic (ROC) curve for the discrimination of rejection from no rejection.

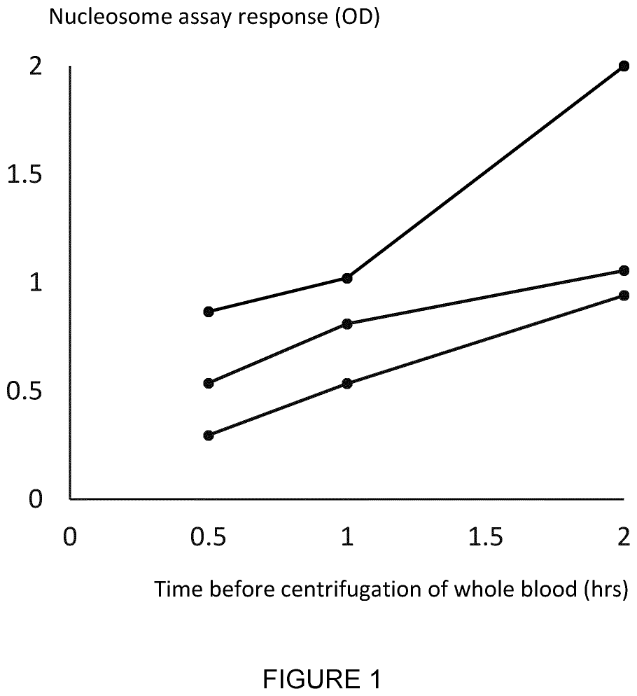

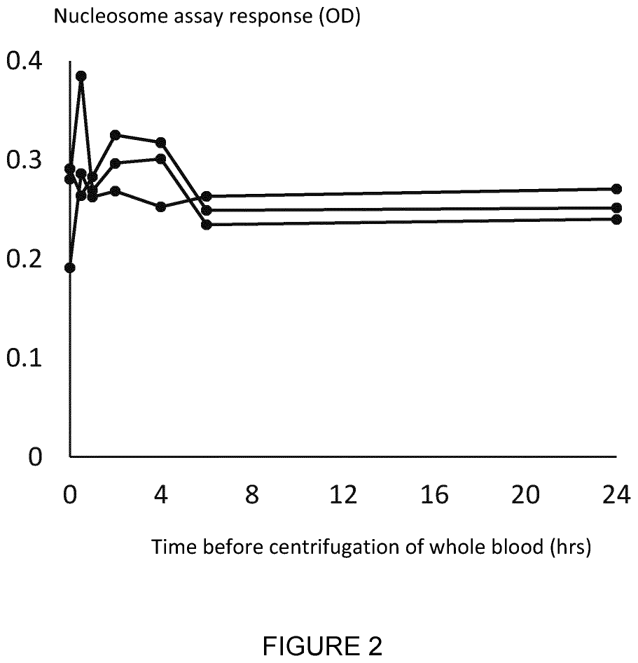

The authors have found that neutrophils in whole blood samples taken into serum blood collection tubes (BCTs), undergo strong time dependent NETosis in the tube in vitro stimulated by coagulation (i.e. NETs are formed ex vivo in the tube as an artifact of serum collection). We measured the level of nucleosomes containing histone variant H3.1 (H3.1-nucleosomes) as a measure of NETs in serum samples collected from 3 healthy subjects and found that the assay response more than doubled if the delay before sample processing was increased from 30 minutes to 2 hours (FIG. 1). Therefore, the nucleosome result obtained for a serum sample is critically and quantitatively dependent on the sample processing delay. As sample processing is routinely performed anywhere between 30 minutes and 8 hours post venepuncture, and the level measured is greatly affected by the delay, serum nucleosome measurements are a poor measure of the true circulating level of cell-free nucleosomes, NETs or ETs. As coagulation does not occur in plasma BCTs and we observed no analogous rise in signal EDTA plasma samples even after 24 hours (FIG. 2), the inventors propose using plasma (e.g. EDTA plasma) in place of serum for improved discrimination and prediction of rejection.

Therefore, in one embodiment the sample is a plasma sample including a plasma sample produced using a calcium sequestrator such as EDTA plasma or citrate plasma.

In one embodiment whole blood is collected in plasma BCT in which the cellular component is stabilized through use of a cross-linking agent. Such BCTs are available commercially such as the Cell-Free DNA BCT available from Streck Inc. These BCTs have the advantage of minimizing chromatin release from dead or dying blood cells in the sample ex vivo post venipuncture and therefore reducing chromatin contamination.

### Biomarkers for Extracellular Traps or Neutrophil Extracellular Traps

Any assay for ETs or NETs may be used including, without limitation, cell-free DNA (cfDNA) or any measurement of chromatin or chromatin fragments including cell-free nucleosomes. It has previously found that circulating cell-free nucleosomes are a highly useful measurement of circulating levels of NETs in sepsis and COVID-19 patients. Therefore, in a preferred embodiment the biomarker used as a measure of the level of ETs or NETs is a nucleosome. It will be understood that the whole nucleosome need not be measured and that a part of a nucleosome may be measured, including cfDNA and (free) histones.

A number of proteins occur in NETs that are adducted directly or indirectly to nucleosomes. These proteins include, without limitation, myeloperoxidase (MPO), neutrophil elastase (NE), lactotransferrin, azurocidin, cathepsin G, leukocyte proteinase 3, lysozyme C, neutrophil defensin 1, neutrophil defensin 3, myeloid cell nuclear differentiation antigen, S100 calcium-binding protein A8, S100 calcium-binding protein A9, S100 calcium-binding protein A12, actin β, actin γ, alpha-actin, plastin-2, cytokeratin-10, catalase, alpha-enolase and transketolase (Urban et al., PLOS Pathogens. (2009) 10: e1000639). Any nucleosome-protein adduct that occurs in NETs is a useful adduct for the detection of elevated levels of NETs.

In one embodiment, the level of ETs or NETs is measured by measuring the level of one or more moieties selected from cell-free DNA or a histone moiety.

In one embodiment, the level of ETs or NETs is measured by measuring the level of nucleosomes per se present in the sample. In an alternative embodiment, the level of ETs or NETs is measured by measuring the level of one or more component(s) of cell free nucleosomes present in the sample. In a further embodiment, the one or more component(s) comprise an epigenetic feature of cell free nucleosomes.

For example, NETs are known to include nucleosomes comprising citrullinated histones (i.e. post translationally modified nucleosomes). Therefore, in one embodiment the epigenetic feature (i.e. nucleosome biomarker) is a citrullinated histone.

As well as neutrophil cell death associated with NETosis, the degeneration and loss of function of an donor organ may also be associated with other elevated cell death processes such as necrosis, apoptosis and others. These forms of cell death are associated with nucleosome release into the extracellular environment. The measurement of nucleosomes as a biomarker or monitor of organ health therefore has a dual advantage of detecting either or both an excessive inflammatory environment with elevated NETs release and also elevated cell death. Therefore, in a most preferred embodiment the biomarker used for the purposes of the invention is a cell free nucleosome.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, Methods Mol. Biol. (2007) 361:25-62).

References to “nucleosome” may refer to “cell free nucleosome” when detected in body fluid samples. It will be appreciated that the term cell free nucleosome throughout this document is intended to include any cell free chromatin fragment that includes one or more nucleosomes.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

Circulating cell free nucleosomes may conveniently be measured by immunoassay as well as by other methods. Any circulating cell free nucleosome measurement may be used for the purposes of the invention including nucleosomes containing particular epigenetic signals including particular post-translational modifications, histone isoforms/variants, modified nucleotides and non-histone chromatin proteins. Various nucleosome types have been measured (as referenced in WO2005019826, WO2013030577, WO2013030579 and WO2013084002 which are herein incorporated by reference).

Methods of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells.

The biomarker used in the methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone isoform, a modified nucleotide and/or proteins bound to a nucleosome in a nucleosome-protein adduct.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone variants can be classed into a number of families which are subdivided into individual types. The nucleotide sequences of a large number of histone variants are known and publicly available for example in the National Human Genome Research Institute NHGRI Histone Database (Marino-Ramĩrez et al. The Histone Database: an integrated resource for histones and histone fold-containing proteins. Database Vol.2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2, H3.3 and H3t.

In one embodiment, the histone isoform is H3.1.

The structure of nucleosomes can vary by post translational modification (PTM) of histone proteins. PTM of histone proteins typically occurs on the tails of the core histones and common modifications include acetylation, methylation or ubiquitination of lysine residues as well as methylation or citrullination of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be present on a core nucleosome histone (e.g. H2A, H2B, H3 or H4), or a linker histone (e.g. H1 or H5). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826 and WO 2017/068359.

For example, the post translational modification may include acetylation, methylation, which may be mono-, di-or tri-methylation, phosphorylation, ribosylation, citrullination, ubiquitination, hydroxylation, glycosylation, nitrosylation, glutamination and/or isomerisation (see Ausio (2001) Biochem Cell Bio 79:693). In one embodiment, the histone PTM is methylation of a lysine residue. In a further embodiment, the methylation is of a histone 3 lysine residue. In a yet further embodiment, the histone PTM is selected from H3K4Me, H3K4Me2, H3K9Me, H3K9Me3, H3K27Me3 or H3K36Me3. In one embodiment, the histone PTM is acetylation of a lysine residue. In a further embodiment, the acetylation is of a histone 3 lysine residue. In a yet further embodiment, the histone PTM is selected from H3K9Ac, H3K14Ac, H3K18Ac or H3K27Ac. In another embodiment, the histone PTM is H4PanAc. In one embodiment, the histone PTM is phosphorylation of a serine residue. In a further embodiment, the phosphorylation is of an isoform X of histone 2A (H2AX) serine residue or phosphorylation of a histone 3 serine residue. In a yet further embodiment, the histone PTM is selected from pH2AX or H3S10Ph. In one embodiment, the histone PTM is selected from citrullination or ribosylation. In a further embodiment, the histone PTM is citrullinated H3 (H3cit) or citrullinated H4 (H4cit). In a further embodiment, the histone PTM is citrullination of a histone 3 arginine residue. In a yet further embodiment, the histone PTM is H3R8Cit. In one embodiment, the histone PTM is selected from the group consisting of: H3K4Me, H3K4Me2, H3K9Me, H3K9Me3, H3K27Me3, H3K36Me3, H3K9Ac, H3K14Ac, H3K18Ac, H3K27Ac, H4PanAc, pH2AX, H3S10Ph and H3R8Cit.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody [H4panAc]), anti-citrullination antibodies or anti- ubiquitin antibodies.

In one embodiment, the histone PTM is selected from citrullination or ribosylation, in particular citrullination. In a further embodiment, the histone PTM is H3 citrulline (H3cit) or H4 citrulline (H4cit). In a yet further embodiment, the histone PTM is H3cit.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

Another way the structure of nucleosomes may vary is by mutation. Therefore, in one embodiment, the epigenetic feature is a mutated histone. In a further embodiment, the mutation is in histone 3 (H3). In a yet further embodiment, the mutation in H3 is when lysine 27 is replaced by a methionine (H3K27M).

It will be understood that more than one epigenetic feature of cell free nucleosomes may be detected in methods and uses of the invention. Multiple biomarkers may be used as a combined biomarker. Therefore, in one embodiment, the method comprises detecting more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected. In one aspect, the use comprises a post-translational histone modification and a histone isoform as a combined biomarker in a sample, for the diagnosis, detection, treatment selection, prognostication or monitoring of an infection. In one embodiment, the combined biomarker is H3.1 and H3cit. In an alternative embodiment, the combined biomarker is H3.1 and H4cit.

### Measurement Methods

As described herein, the methods of the invention are preferably performed in vitro. In one embodiment, the method of measuring (i.e. measuring the level of ETs or NETs in the sample) comprises contacting the sample with a solid phase comprising a binding agent that detects cell free nucleosomes or a component thereof, and detecting binding to said binding agent.

In a further embodiment, the method of measuring comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

In a further embodiment, the method of measuring comprises: (i) contacting the sample with a first binding agent which binds to cell free nucleosomes; (ii) contacting the sample bound by the first binding agent in step (i) with a second binding agent which binds to an epigenetic feature of a cell free nucleosome; and (iii) detecting or quantifying the binding of the second binding agent in the sample.

It will be understood that any first and second binding agents may be added to the sample separately in any order or may be added simultaneously. Furthermore, the method may be repeated on one or more occasions and any changes in the level of binding to the first binding agent and/or the second binding agent is used to monitor the status of the donor organ or tissue.

In one embodiment, the level or concentration of ETs or NETs in the sample detected is compared to a control or a reference level. It will be clear to those skilled in the art that the control organs or subjects may be selected based on a variety of factors which may include, for example, organs or subjects known to be healthy. The “control” may comprise a healthy subject, a non-diseased subject and/or an otherwise healthy subject with a transplanted organ. The control may also be a healthy extracorporeal organ. Comparison with a control is well known in the field of diagnostics.

It will be understood that it is not necessary to measure controls for comparative purposes on every occasion because once the ‘normal range’ or ‘acceptable range for transplantation’ is established it can be used as a benchmark for all subsequent tests. A normal range can be established by obtaining samples from multiple control subjects or organs and testing for the level of biomarker. Results (i.e. biomarker levels) for test organs or subjects can then be examined to see if they fall within, or outside of, the respective acceptable or normal range. Use of a such ranges is standard practice in diagnostic testing.

In one embodiment, the level or concentration of ETs or NETs is elevated compared to a control or reference level. In methods in which more than one biomarkers are determined these levels may be compared to a reference wherein the levels or ratios of the plurality of biomarkers in comparison to the reference levels or ratios is indicative of the health or suitability for transplant of a donor organ or tissue.

In other words, the level of the biomarker is compared to a reference level. Reference to a “reference level” refers to a level of biomarker concentration in a sample against which a test sample is compared.

If a subject or organ is determined to be healthy or acceptable for transplantation, then the invention may still be used for the purposes of monitoring continued organ and/or subject health. For example, if the use comprises a plasma sample from a subject determined to have a stable or healthy organ graft, then the biomarker level measurements can be repeated at another time point to establish if the biomarker level has changed.

Therefore, once a donor organ has been transplanted into a recipient subject in need of an organ, the recipient subject must be further monitored for the health of the transplanted organ and for organ rejection. The methods of the invention are low cost, high throughput, require a fraction of the blood volume needed for cfDNA sequencing and may be performed rapidly to inform patient management. Therefore, methods of the invention may be used to measure nucleosomes in blood, serum, plasma or other body fluid samples obtained from the organ recipient, as an indicator of tissue damage and cell death of the transplanted organ and risk of rejection. This method will be particularly suitable for use with kidney, liver, lung and heart transplantation. Therefore, in one embodiment, the donor organ is selected from a kidney, liver, heart, lung, pancreas, stomach or intestine. In one embodiment, the donor tissue is selected from whole blood, plasma, platelets, cornea, bone, tendon, skin, pancreas islets, heart valves, nerves, veins, bone marrow or stem cells. In one embodiment, the donor tissue is a limb, such as a hand, an arm or a foot.

References to “subject”, “individual” or “patient” are used interchangeably herein. In one embodiment, the subject or donor organ or donor tissue, is a human subject, human donor organ or human donor tissue. In one embodiment, the subject or donor organ or donor tissue is a (non-human) animal subject or animal donor organ or animal donor tissue. In some embodiments the invention encompasses animal subjects (wild or domesticated). In some embodiments, the invention relates to veterinary uses including for livestock and companion animals such as cats, dogs, horses, sheep, goats, pigs, deer, llamas, cows and cattle.

In one embodiment the donor and recipient subjects are not selected from individuals of the same species. For example, a human subject may be the recipient of an organ graft donated by a subject of another species (for example, a pig).

The methods described herein are preferably performed in vitro. References to acts carried out on a body fluid sample “obtained” from a subject are intended to encompass acts carried only a body fluid sample already obtained of “obtainable” from a subject and vice versa.

Cell free nucleosomes in a body fluid sample may conveniently be measured by immunoassay as well as by other immunochemical methods, mass spectrometry and other analytical methods. Any analytical method for the measurement of cell free nucleosomes in a body fluid may be used for the purposes of the invention. In one embodiment, detection or measurement of the biomarker(s) comprises an immunoassay, immunochemical, mass spectroscopy, chromatographic, chromatin immunoprecipitation or biosensor method. In one embodiment, the method is performed using immunoassay. In another embodiment, the method is performed using chromatin immunoprecipitation.

In one embodiment, the detection or measurement comprises an immunoassay. In a preferred embodiment of the invention there is provided a 2-site immunoassay method for nucleosome moieties. In particular, such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. In another embodiment of the invention, there is provided a 2-site immunoassay employing a labelled anti-nucleosome detection binding agent in combination with an immobilized anti-histone modification or anti-histone variant or anti-DNA modification or anti-adducted protein binding agent.

In a further embodiment of the invention there is provided a homogeneous immunoassay (HIA) method for the measurement or detection of the level of ETs or NETs in the sample, e.g. in the measurement of nucleosome moieties. In particular, such a HIA method is preferred for the measurement of nucleosomes. HIA is rapid assay methodology requiring only a few minutes to provide a result. The provision of rapid results has the advantage of facilitating rapid appropriate decisions and actions by physicians.

In a further embodiment of the invention there is provided a point of care (POC) method for the measurement or detection of the level of ETs or NETs in the sample, e.g. the level of nucleosome moieties. In particular, such a POC method is preferred for the measurement of nucleosomes. POC methodology provides rapid results directly to the physician by obviating the necessity for sending samples to a laboratory for analysis and waiting on the results. The provision of POC results has the advantage of facilitating rapid appropriate decisions and actions by physicians. An example of a POC system includes the fluorescent immunoassay system of LightDeck Diagnostics.

Detecting or measuring the level of the biomarker(s) may be performed using one or more reagents, such as a suitable binding agent. In one embodiment, the binding agent comprises a ligand or binder specific for the desired biomarker, e.g. ETs or NETs. The term “biomarker” as defined herein includes any single biomarker moiety or a combination of individual biomarker moieties in a “biomarker panel”.

It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention. In one embodiment, the binding agent, such as the antibody, specifically binds to the target biomarker. The specificity of an antibody is the ability of the antibody to recognize a particular antigen as a unique molecular entity and distinguish it from another. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances.

Methods of detecting biomarkers are known in the art, e.g. using one or more ligands or binders. In one embodiment, the ligands or binders include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention there is provided a kit for: the assessment of the health or suitability for transplant of an extracorporeal organ or tissue; assessment of the health of a donated organ or tissue graft; or prediction or assessment of the risk of the rejection of a grafted organ or tissue, comprising a biosensor capable of detecting and/or quantifying one or more of the biomarkers as defined herein. Optionally, said kit may comprise instructions for use. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention. According to a further aspect of the invention, there is provided a kit for assessing the health or suitability for transplant of an extracorporeal organ or tissue (as described herein), wherein said kit comprises a first binding agent which specifically binds to an epigenetic feature of a cell free nucleosome (e.g. H3.1) and a second binding agent which specifically binds to cell free nucleosomes.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Biomarkers for detecting the degeneration of organ health are essential targets for discovery of novel targets and drug molecules that retard or halt progression of organ degeneration or organ rejection. As the result for a biomarker or biomarker panel is indicative of disorder and of drug response, the biomarker is useful for identification of novel therapeutic compounds in in vitro and/or in vivo assays. Biomarkers and biomarker panels of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The term “detecting” or “diagnosing” as used herein encompasses identification, confirmation, and/or characterisation of a disease state, degeneration state or health state of an individual or an organ. Methods of detecting, monitoring and of diagnosis according to the invention are useful to confirm the health or condition of a subject or an organ, to monitor development of the condition by assessing onset and progression, or to assess amelioration or regression of the condition. Methods of detecting, monitoring and of diagnosis are also useful in methods for assessment of clinical screening, prognosis, choice of therapy, evaluation of therapeutic benefit, i.e. for drug screening and drug development.

Identifying and/or quantifying can be performed by any method suitable to identify the presence and/or amount of a specific protein in a biological sample from a subject or a purification or extract of a biological sample or a dilution thereof. In methods of the invention, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted, in particular, that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein.

Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels.

In another example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

Biomarker monitoring methods, biosensors and kits are also vital as subject monitoring tools, to enable the physician to determine whether rejection of the organ or tissue is likely to occur. If uptake of the donor organ or tissue is assessed to be inadequate, then therapy (e.g. immunosuppressive drug treatment to reduce or slow graft rejection) can be reinstated or increased; a change in therapy can be given if appropriate. As the biomarkers are sensitive to the state of the donor organ health, they provide an indication of the impact of therapy.

According to a further aspect of the invention, there is provided a method of treating transplant rejection in a transplant recipient, comprising:
(i) obtaining a plasma sample from the transplant recipient;
(ii) measuring the level of ETs or NETs in the plasma sample;
(iii) using the level of ETs or NETs measured as an indicator of the risk of rejection of a donor organ or tissue in the transplant recipient; and
(iv) administering an immunosuppressive drug if the transplant recipient is determined in step (iii) to be at risk of rejecting the donor organ or tissue.

The term “immunosuppressive drug” includes any drug that suppresses, or inhibits, the activity of the patient's immune system. Such drugs include, without limitation, calcineurin inhibitors (such as cyclosporine or tacrolimus), anti-proliferative agents (such as mycophenolate mofetil or azathioprine), mTOR Inhibitors (such as sirolimus and everolimus), corticosteroids (such as prednisolone or methylprednisolone), or monoclonal antibodies (such as Basiliximab or Alemtuzumab). In one embodiment, the immunosuppressive drug is selected from tacrolimus, cyclosporine, mycophenolate mofetil, azathioprine, everolimus, sirolimus, and glucocorticoids (e.g. steroids).

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Example 1

Whole blood from 3 healthy subjects was each collected into 3 serum blood collection tubes (BCTs). The BCTs were left at room temperature for 0.5, 1 or 2 hours respectively and then centrifuged to pellet the cellular fraction of the blood and the supernatant serum was removed and frozen until assay. The serum samples were then assayed for H3.1-nucleosomes using a manual enzyme linked immunosorbent assay (ELISA). In brief, 80 μl assay buffer and 20 μl of plasma sample was added to a microtiter well coated with an antibody directed to bind to histone H3.1. The microtiter plate was covered and incubated at room temperature with gentle shaking. The contents of the microtiter wells were discarded and the wells were washed three times with 200 μl of a wash solution. 100 μl of a labelled anti-nucleosome antibody was added and the microtiter plate was again covered and incubated. The contents of the microtiter wells were then again discarded and the wells were washed. Bound labelled antibody was developed using 100 μl of a HRP (horse radish peroxidase) substrate solution and the absorbance (OD) of the wells was measured at 405 nm.

The results (FIG. 1) show that measured serum levels of H3.1 nucleosomes increases with the time whole blood is left prior to processing by centrifugation. This is due to coagulation induced NETosis by neutrophils in the sample ex vivo and indicates that H3.1-nucleosome levels measured in serum are critically dependent on the time whole blood is stored prior to processing. As the time left before processing may vary between 20 minutes to 24 hours, it is clear that serum is not an optimal matrix for nucleosome or NETs measurements.

### Example 2

A similar experiment to that conducted in EXAMPLE 1 for serum was performed using EDTA plasma BCTs. Whole blood collected from 3 healthy volunteers in EDTA BCTs was centrifuged immediately and at 30 minutes, 1 hour, 2 hours, 4 hours, 6 hours and 24 hours following venepuncture. The level of plasma NETs was again measured as the plasma level of H3.1-nucleosomes. The assay response (Optical Density), corresponding to the plasma H3.1-nucleosome level, did not increase over 24 hours indicating that no NETosis occurred ex vivo in EDTA plasma samples and that EDTA plasma nucleosome measurements are a valid measure of circulating NETs and nucleosomes.

1. A method for the assessment of the health or suitability for transplant of a donor organ or tissue, wherein said method comprises the steps of:
(i) obtaining a fluid sample from the donor organ or tissue;
(ii) measuring the level of extracellular traps (ETs) or neutrophil extracellular traps (NETs) in the sample; and
(iii) using the level of ETs or NETs measured as an indicator of the health of the donor organ or tissue or the suitability for transplant of the donor organ or tissue.

2. A method for the assessment of the health of a donor organ or tissue that has been transplanted in a subject wherein said method comprises the steps of:
(i) obtaining a plasma sample from the subject;
(ii) measuring the level of ETs or NETs in the plasma sample; and
(iii) using the level of ETs or NETs measured as an indicator of the health of a donor organ or tissue in the subject.

3. A method for the prediction or assessment of the risk of rejection of a donor organ or tissue that has been transplanted in a subject wherein said method comprises the steps of:
(i) obtaining a plasma sample from the subject;
(ii) measuring the level of ETs or NETs in the plasma sample; and
(iii) using the level of ETs or NETs measured as an indicator of the risk of rejection of the donor organ or tissue in the subject.

4. The method of claim 1, wherein the donor organ is selected from the group consisting of a kidney, a liver, a heart, a lung, a pancreas, a stomach and an intestine.

5. The method of claim 1, wherein the donor tissue is selected from the group consisting of whole blood, plasma, platelets, cornea, bone, tendon, skin, pancreas islets, heart valves, nerves, veins, bone marrow and stem cells.

6. The method of claim 1, wherein the donor tissue is a limb, such as a hand, an arm or a foot.

7. The method of claim 1, the level of ETs or NETs is measured by measuring the level of one or more component(s) of cell free nucleosomes present in the sample.

8. The method of claim 7, wherein the one or more component(s) comprise an epigenetic feature of cell free nucleosomes.

9. The method of claim 8, wherein the epigenetic feature is a citrullinated histone.

10. The method of claim 8, wherein the epigenetic feature is a histone isoform.

11. The method of claim 10, wherein the histone isoform is H3.1.

12. The method of claim 1, wherein the level of ETs or NETs is measured by measuring the level of nucleosomes per se present in the sample.

13. The method of claim 1, wherein the level of ETs or NETs is measured using a technique selected from the group consisting of an immunoassay, an immunochemical method, mass spectroscopy, a chromatographic method, chromatin immunoprecipitation and a biosensor method.

14. The method of claim 1, wherein the method of measuring comprises contacting the sample with a solid phase comprising a binding agent that detects cell free nucleosomes or a component thereof, and detecting binding to said binding agent.

15. The method of claim 1, wherein the method of measuring comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

16. The method of claim 1, wherein the donor organ or tissue is obtained from a human or an animal subject.

17. The method of claim 2, wherein the subject is a human or an animal subject.

18. The method of claim 1, further comprising comparing the level of ETs or NETs in the sample with one or more controls.

19. The method of claim 3, wherein the method of measuring comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

20. The method of claim 19, wherein the epigenetic feature is histone isoform H3.1.
